(19) United States
(12) Patent Application Publication
(10) Pub. No.: US 2009/0064802 A1
(43) Pub. Date: Mar. 12, 2009

(54) Method for Measuring Sample, Package for Sensor Chips, and Mechanism for Fixing Sensor Chips

(76) Inventors: Kenichi UCHIYAMA, Chigasaki-shi (JP); Tomohiro Takase, Sagamihara-shi (JP); Ikuo Uematsu, Yokohama-shi (JP); Shingo Kasai, Yokohama-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/206,032

(22) Filed: Sep. 8, 2008

(30) Foreign Application Priority Data
Sep. 10, 2007 (JP) P2007-234285

(51) Int. Cl. G01N 33/00; H01L 29/00; B23P 19/00

(52) U.S. Cl. 73/866; 257/414; 29/740; 257/E29.001

(57) ABSTRACT

A method for measuring samples includes fitting and mounting, onto a transfer stage, a chip pallet for transporting sensor chips; fitting a positioning hole of a chip package onto a positioning pin; pulling a bottom plate of the chip package off the chip package, so as to simultaneously transfer the housed sensor chips onto the transfer stage; pulling upward the chip pallet so as to simultaneously transfer, onto the chip pallet, the sensor chips on the transfer stage; and mounting the chip pallet having the sensor chips mounted thereon onto a measuring device, and measuring samples.

### CROSS REFERENCE TO RELATED APPLICATIONS

This application is based upon and claims the benefit of priority from prior Japanese Patent Application 2007-234285 filed on Sep. 10, 2007 the entire contents of which are incorporated by reference herein.

### BACKGROUND OF THE INVENTION

1. Field of the Invention

The present invention relates to a method for measuring a sample in a sensor chip, a chip package for housing sensor chips, and a mechanism for fixing sensor chips.

2. Description of the Related Art

In the fields of clinical laboratory science and environmental measurement, devices capable of obtaining test results in a short time are now actively developed. Examples of such devices include sensor chips for immunochromatographic systems, dry-chemistry systems, μTAS systems, and the like.

Such sensor chip is housed in a package including: a bottom member having a depression (a pocket) formed by vacuum forming; and a film for covering the pocket. When housed in the package, the sensor chip is placed inside the pocket in a manner that a reservoir for causing reaction of a sample therein of the sensor chip faces upward. The sensor chip is then covered with the film from above. Placing the sensor chip inside the pocket in this manner makes it possible to effect the reaction of a sample in the reservoir of the sensor chip only by removing the film, with the bottom member serving as a tray for the sensor chip. The sample placed in the reservoir is caused to react for a certain period of time. Thereafter, the chip is transferred onto a measuring device for optically or electrically reading signals, so as to obtain a result of measurement.

Many measuring devices to be used for such measurement are configured to measure a single sensor chip, and the operator manually transfers the sensor chip to the measuring device. Meanwhile, there are measuring devices capable of having multiple sensor chips mounted together thereon for allowing a large number of samples to be measured in a short period of time, as disclosed in Japanese Patent Application Publication No. 2006-153642. However, even in such measuring device, the operator manually transfers sensor chips one by one onto the measuring device.

The sensor chips thus transferred are pressed by a spring against pins serving as references for positioning, and are thus fixed, for the purpose of facilitating the reading of the sample. In a case where the sensor chips are simply placed on a stage of a measuring device without precise positioning of the sensor chips, an optical system for reading signals scans over a wide range so as to read the samples.

However, the following problems arise if the operator transfers multiple sensor chips one by one onto a measuring device as disclosed in Japanese Patent Application Publication No. 2006-163642. Specifically, such transferring operation is very laborious and inefficient in any way, for example, where the operator uses a tool such as tweezers, or where the operator directly holds and transfers the sensor chips.

Moreover, in some cases, the sensor chips contain, for example, hazardous samples such as viruses, and need to be handled with care. In this case, extra attention is required particularly, for example, when the operator directly holds the sensor chips for transferring.

Meanwhile, many sensor chips, for example, many optochemical sensor chips, include bases made of glass or quartz. Consider using, for such sensors, a measuring device having a mechanism for pressing a sensor chip by a spring against a reference pin for the positioning of the sensor chip. In this case, the sensor chips may possibly be damaged, for example, a part, coming into contact with the reference pin, of the sensor chip may be cracked. Particularly, if the reference pin is made of a metal, the possibility of such damage is increased. Also consider using a measuring device in which an optical system has a mechanism for scanning sensor chips over a wide range without much precision in the positioning of the sensor chips. Such measuring device tends to have a complicated scanning mechanism as compared with measuring devices capable of highly-precise positioning.

### BRIEF SUMMARY OF THE INVENTION

A first aspect of the present invention is a method for measuring a sample, including fitting and mounting, onto a transfer stage, a frame-shaped chip pallet for transporting a plurality of sensor chips, the transfer stage being provided in a chip transfer block; fitting a positioning hole of a chip package having the sensor chips housed therein, on a positioning pin provided on the chip transfer block, so as to position the chip package, in a longitudinal direction, on the transfer stage; pulling, off the chip package, a bottom plate thereof which holds lower surfaces of the sensor chips, so as to simultaneously transfer the sensor chips respectively onto positions each between bars for preventing the sensor chips from moving, the bars being formed on a mounting face, onto which the sensor chips are mounted, of the transfer stage; pulling upward the chip pallet to bring the chip pallet into contact with four corners of each of the sensor chips mounted on the transfer stage, so that the sensor chips are simultaneously transferred onto the chip pallet; and mounting the chip pallet having the sensor chips mounted thereon onto a measuring device, and measuring a sample in the sensor chips.

A second aspect of the present invention is a chip package including: a housing body having a plurality of chip pockets formed at equal intervals, the chip pockets respectively housing a plurality of sensor chips each having a sample injection portion formed in an upper surface thereof the chip pockets having an opening portion exposing the sample injection portions to the outside; and a bottom plate for holding lower surfaces of the respective sensor chips. In the chip package, the bottom plate and the housing body are assembled in a manner that the bottom plate is slidable along and between folded-back portions formed in the housing.

A third aspect of the present invention is a method for measuring a sample, including: mounting a chip-discharging pallet onto a chip-fixing block of a measuring device for reading samples in a plurality of sensor chips; fitting a positioning hole of a chip package, which houses the sensor chips, onto a positioning pin provided on the chip-fixing block, so as to set, on the transfer stage, a position, in a longitudinal direction, of the chip package; pulling a bottom plate, which holds lower surfaces of the sensor chips, off the chip package, so as to simultaneously transfer the sensor chips respectively into mounting pockets formed in the chip-discharging pallet; aligning the sensor chips by displacing the positions of the sensor chips mounted in the mounting pockets by use of a displacement mechanism provided to the measuring device; fixing, by use of a fixing jig, the sensor chips aligned in the mounting pockets at the positions of the respective sensor chips; and measuring the samples in the fixed sensor chips.

A fourth aspect of the present invention is a chip-fixing device including: a chip-fixing block having guides for mounting a plurality of sensor chips; a displacement mechanism for displacing the positions of the respective sensor chips by moving the chip-fixing block; and a fixing jig for fixing the positions of the sensor chips displaced on the chip-fixing block. The displacement mechanism includes: a cam mechanism including a first cam and a second cam, the first cam moving a first follower in one direction, the second cam being connected to the first follower and moving a second follower in a direction orthogonal to the direction in which the first follower moves, the second follower being connected to the chip-fixing block; and a first linear guide bearing being connected to the second follower, and moving the chip-fixing block in a direction not orthogonal to the direction in which the second follower moves. The fixing jig includes: a third follower moved by the first cam in the same direction as that in which the first follower moves; a third cam connected to the third follower; a fourth follower moved by the third cam in the same direction as that in which the third follower; a second linear guide bearing allowing the fourth follower to move in a direction orthogonal to the sensor chips mounted on the chip-fixing block; and chip-fixing pins connected to the fourth follower in an orthogonal direction, and are brought respectively into contact with the sensor chips so as to fix the sensor chips.

### BRIEF DESCRIPTION OF THE SEVERAL THE DRAWINGS

FIG. 1 is a plan view showing an appearance of a chip package according to embodiments of the present invention.

FIG. 2 is a plan view showing a housing body of the chip package according to the embodiments of the present invention.

FIG. 3 is a cross-sectional view showing the chip package shown in FIG. 1, and taken along the line A-A.

FIG. 4 is a cross-sectional view showing the chip package shown in FIG. 1, and taken along the line B-B.

FIG. 5 is a cross-sectional view showing the chip package shown in FIG. 1, and taken along the line C-C.

FIG. 6 is a cross-sectional view showing the chip package shown in FIG. 1, and taken along the line D-D.

FIG. 7 is a cross-sectional view showing the chip package shown in FIG. 1, and taken along the line E-E.

FIG. 8 is a plan view showing an appearance of a bottom plate of the chip package according to the embodiments of the present invention.

FIG. 9 is a plan view showing the chip package, according to the embodiments of the present invention, in which a sensor chip is housed.

FIG. 10 is a cross-sectional view showing the chip package shown in FIG. 9, and taken along the line F-F.

FIG. 11 is a cross-sectional view briefly showing, by cutting the chip package in the longitudinal direction, a state where the chip package housing sensor chips is fixed with a positioning pin of a chip transfer block inserted therethrough.

FIG. 12 is a cross-sectional view briefly showing how the sensor chips fall down when the bottom plate is pulled off from the state shown in FIG. 11.

FIG. 13 is a perspective view showing a chip pallet according to the embodiments of the present invention.

FIG. 14 is a perspective view showing a chip transfer block according to the embodiments of the present invention.

FIG. 15 is an explanatory view showing a first transfer process of the sensor chips according to a first embodiment of the present invention.

FIG. 16 is an explanatory view showing a second transfer process of the sensor chips according to the first embodiment of the present invention.

FIG. 17 is an explanatory view showing a third transfer process of the sensor chips according to the first embodiment of the present invention.

FIG. 18 is an explanatory view showing a fourth transfer process of the sensor chips according to the first embodiment of the present invention.

FIG. 19 is an explanatory view showing a fifth transfer process of the sensor chips according to the first embodiment of the present invention.

FIG. 20 is an explanatory view showing a sixth transfer process of the sensor chips according to the first embodiment of the present invention.

FIG. 21 is an overall view showing a chip-fixing block, a chip-discharging pallet, a displacement mechanism, and a fixing jig, all used in a transfer process according to a second embodiment of the present invention.

FIG. 22A is a plan view showing the chip-discharging pallet according to the second embodiment of the present invention.

FIG. 22B is a cross-sectional view showing the chip-discharging pallet according to the second embodiment of the present invention, and taken along the line G-G.

FIG. 23 is a schematic view showing a relationship between a cam mechanism and first linear guide bearings according to the second embodiment of the present invention, and more specifically, showing a relationship between a second follower and the first linear guide bearings.

FIG. 24 is a schematic view showing the fixing jig having reached the position above the sensor chips) according to the second embodiment of the present invention, as viewed in the X-axis direction.

FIG. 25 is a schematic view for explaining the movement and fixation of the sensor chips in association with the movement of the displacement mechanism.

FIG. 26 is another schematic view for explaining the movement and fixation of the sensor chips in association with the movement of the displacement mechanism.

### DETAILED DESCRIPTION OF THE INVENTION

Hereinafter, embodiments of the present invention will be described in detail with reference to the drawings.

### First Embodiment

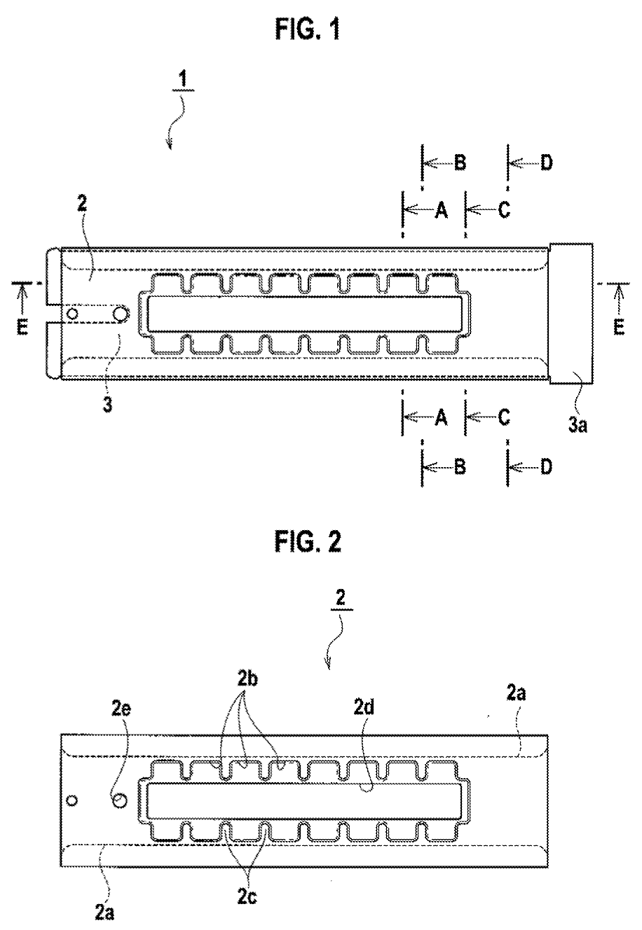

A chip package 1 for housing sensor chips according to an embodiment of the present invention has an appearance as shown in a plan view of FIG. 1, for example. The chip package 1 is formed of a housing body 2 for housing sensor chips SC therein, and a bottom plate 3. The bottom plate 3 is sandwiched and held by a pair of folded-back portions 2a and 2a of the housing body 2 so as to form a bottom plate of the chip package 1. Thus, the bottom plate 3 holds the sensor chips SC to be housed, in a manner that the lower surfaces of the sensor chips SC are brought into contact with the bottom plate 3. The bottom plate 3 is designed to be moved between the folded-back portions 2a and thus removed from the housing body 2 by gripping and pulling a gripper 3a.

FIG. 2 is a plan view showing only the housing body 2. The housing body 2 is formed by, for example, performing vacuum forming on a polyethylene terephthalate (PET) sheet. The folded-back portions 2a and 2a for holding the bottom plate 3 are formed by, for example, a bending process with hot pressing. In the plan view of the housing body 2 shown in FIG. 2, the folded-back portions 2a and 2a are indicated by the dashed lines because each of the folded-back portions 2a and 2a is folded back to the bottom surface side of the housing body 2.

The housing body 2 has pockets 2b for housing the sensor chips SC. Each of the sensor chips SC housed in the pockets 2b has a sample injection portion formed at the center of an upper portion of a reservoir having the shape of rectangular parallelepiped. For this reason, each of the sensor chips SC has a convex cross-sectional shape. Each of the pockets 2b has a rectangular shape slightly larger than the outer dimensions of each sensor chip SC, so that the sensor chip SC is allowed to be housed in the pocket 2b. Employing the pockets 2b each having such a size prevents the sensor chip SC in the pocket 2b from being shifted to a large extent. In addition, each of the pockets 2b is formed into a shape protruding from the surface of the housing body 2 to a aide opposite to the folded-back portions 2a, in order to house the sensor chip SC having the above-described shape.

The bottom plate 3 is fitted into the folded-back portions 2a and 2a, thus serving as the bottom plate of the chip package 1, so that a space in which the sensor chips SC are housed is formed by the housing body 2 and the bottom plate 3. Then, each sensor chip SC is housed in the corresponding pocket 2b formed in the housing body 2 while the lower surface of the sensor chip SC is held by the bottom plate 3.

Multiple pockets 2b are formed in the housing body 2. Moreover, each of the pockets 2b is formed so as to allow the sensor chip SC to be housed therein in a manner that the longitudinal side of the sensor chip SC intersects, at night angles, the longitudinal side of the housing body 2. The formation of the pockets 2b in the above-described manner makes it possible to house the multiple sensor chips SC in the pockets 2b in a manner that the sample injection portions of the respective sensor chips SC are aligned in a line.

Moreover, each adjacent two of the multiple pockets 2b are divided by a partition wall 2c. These partition walls 2c makes it possible to individually house the sensor chips SC respectively in the pockets 2b, instead of collectively housing the multiple sensor chips SC. In addition, the provision of the partition walls 2c defies a mounting position of each sensor chip SC at the time when the sensor chips SC are transferred to a chip transfer block or a chip pallet, which will be described later. Each of the partition walls 2c lie in the same plane as the surface of the housing body 2, but does not have a shape protruding from the housing body 2 as in the case of the pockets 2b. The partition wall 2c with this shape divides each adjacent two of the pockets 2b from each other.

Although eight pockets 2b are provided while being divided from one another by seven partition walls 2c in the embodiment of the present invention, it may be freely set in consideration of the shape and the like of each sensor chip SC how many pockets 2b are provided in the housing body 2.

An opening portion 2d is formed in the pockets 2b by cutting off parts, at which the sample injection portions of the sensor chips SC are positioned, of the pockets 2b. Through this opening portion 2d, the pockets 2b, formed between the housing body 2 and the bottom plate 3 are opened partially at the positions of the sample injection portions of the respective sensor chips SC housed in the pocket 2b. The opening portion 2d thus formed in this manner makes it possible to perform examination by injecting a sample into the sample injection portion without removing, from the chip package 1, the sensor chips SC housed in the chip package 1.

Before the start of examination, the chip package 1 is handled in a state where the sensor chips SC are housed in the chip package 1. In this state, the opening portion 2d is closed up by a seal or the like attached to the part of the opening portion 2d. In the embodiment of the present invention, the opening portion 2d is formed, as shown in FIG. 1, in such a manner that the part is opened over the plurality of pockets 2b. Alternatively, the opening portion 2d may be formed in such a manner that each of the pocket 2b is individually opened only in a part at the position of the sample injection portion of the corresponding sensor chip SC.

A positioning hole 2e is formed in the surface of the housing body 2. In the embodiment of the present invention, the positioning hole 2e is formed, as shown in FIG. 2, at a position in a vicinity of the pocket 2b located closest to the edge among the multiple pockets 2b. Moreover, as shown in FIG. 2, the positioning hole 2e is formed in such a manner that the center of each of the shorter sides of the opening portion 2d and the center of the positioning hole 2e are positioned on the same axis. The positioning hole 2e is used for positioning the chip package 1 and a chip transfer block 31, which will be described later, when the sensor chips SC housed in the chip package 1 is transferred onto the chip transfer block 31.

Specifically, a positioning pin 34 provided on the chip transfer block 31 is inserted through the positioning hole 2e from the bottom plate 3 side. The position, in the longitudinal direction, of the chip package 1 can thereby be determined with respect to the chip transfer block 31. Note that, although the positioning hole 2e is formed, as described above, at the position shown in FIG. 2 in the embodiment of the present invention, the position of the positioning hole 2e may be set appropriately on the surface of the housing body 2 in accordance with the position of the positioning pin 34 on the chip transfer block 31.

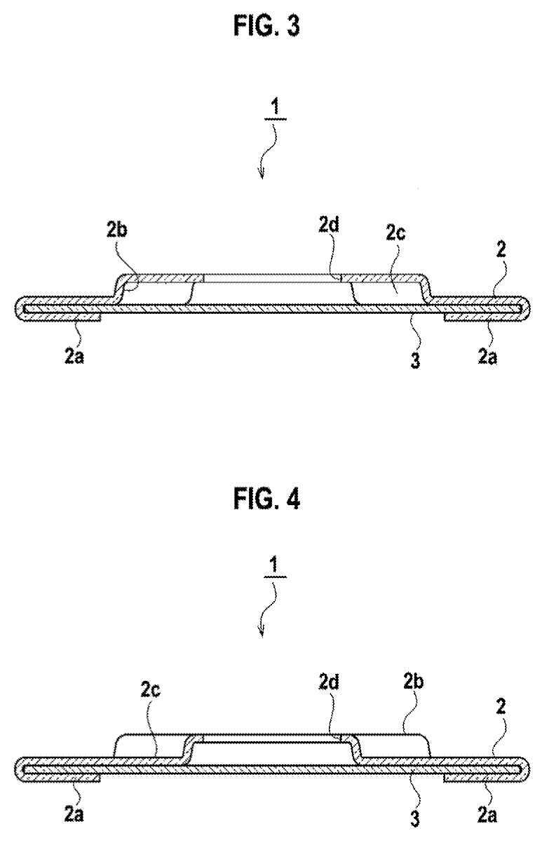

FIG. 3 is a cross-sectional view showing the chip package 1 taken along the line A-A in FIG. 1. In the cross-sectional views including FIG. 3, which are hereinafter referred to, cut surfaces are all indicated by hatching. The housing body 2 is provided with the pair of folded-back portions 2a and 2a, and the bottom plate 3 is mounted on the folded-back portions 2a and 2a. The pockets 2b are formed in a shape protruding from the surface of the housing body 2, so that the sensor chips SC held, at the lower surfaces thereof, by the bottom plate 3 is housed respectively in the pockets 2b. In addition, as shown in FIG. 1, since the line A-A cuts the center of the pocket 2b, the partition walls 2c can be seen in the depth of FIG. 3.

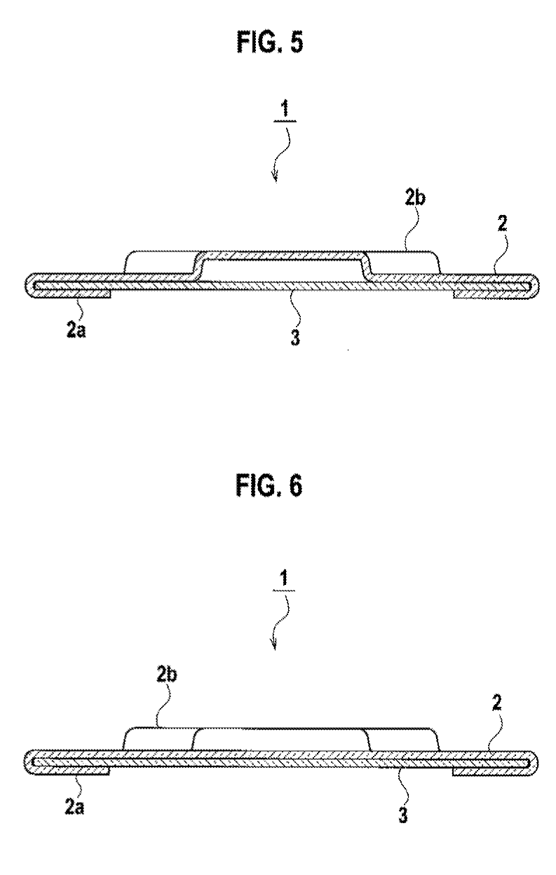

FIG. 4 is a cross-sectional view showing the chip package 1 taken along the line B-B in FIG. 1. The line B-B cuts the part where the partition walls 2c are located. As shown in the cross-sectional view of FIG. 4, the partition walls 2c lie in the same plane as the surface of the housing body 2. Moreover, FIG. 5 is a cross-sectional view showing the chip package 1 taken along the line C-C in FIG. 1. Since the line C-C cuts the part where the end portion of the opening portion 2d formed in the pockets 2b is located, the opening portion 2d is not illustrated in FIG. 5. FIG. 6 is a cross-sectional view showing the chip package 1 taken along the line D-D in FIG. 1. Moreover, it can be seen from FIGS. 3 and 6 that the bottom plate 3 is sandwiched and held by the folded-back portions 2a and 2a, as well as the surface of the housing body 2.

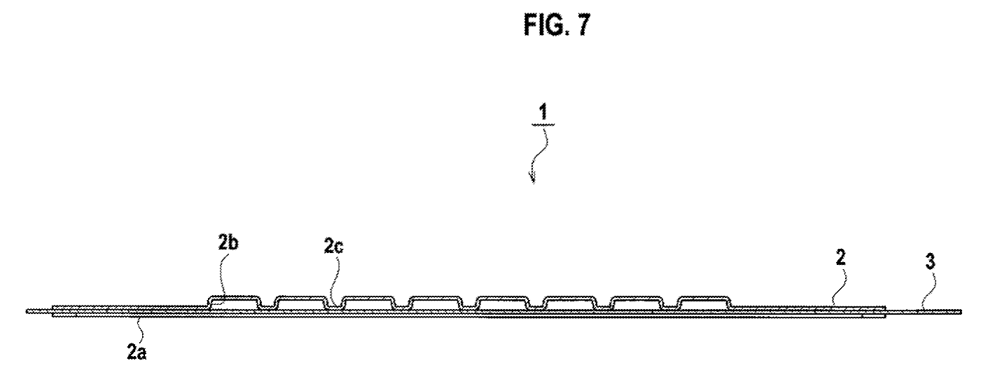

FIG. 7 is a cross-sectional view showing the chip package 1 taken along the line E-E in FIG. 1. It can be seen from FIG. 7 that the multiple pockets 2b are divided by the partition walls 2c so as to be capable of individually housing the sensor chips SC. In addition, while the bottom plate 3 is sandwiched and held by the surface of the housing body 2 as well as the folded-back portions 2a and 2a, and also serves as the bottom plate of the pockets 2b, thereby forming the space for housing the sensor chips SC.

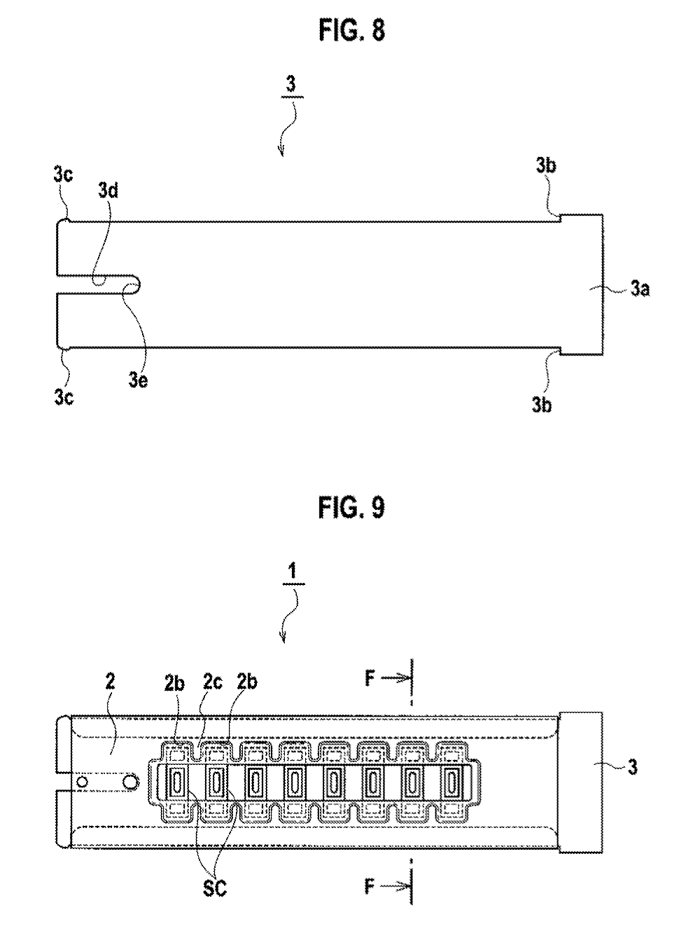

FIG. 8 is a plan view showing the bottom plate 3. The bottom plate 3 has a substantially rectangular shape in the plan view. The gripper 3a is provided at one end portion, in the longitudinal direction, of the bottom plate 3. The gripper 3a is a part to be gripped for pulling the bottom plate 3 in a sliding manner out of the housing body 2 when the sensor chips SC housed in the chip package 1 are transferred to the chip transfer block 31. Moreover, first protruding portions 3b and 3b are formed respectively on the two edge portions of the gripper 3a. When the bottom plate 3 is mounted on the housing body 2, the first protruding portions 3b and 3b come into contact with parts connecting the surface of the housing body 2 to the respective folded-back portions 2a and 2a, thereby preventing the bottom plate 3 from fining off the housing body 2.

Second protruding portions 3c and 3c are formed on the two edges of the other end portion, in the longitudinal direction, of the bottom plate 3. These second protruding portions 3c and 3c have a function of preventing the bottom plate 3 from falling off the housing body 2 in the same manner as that of the first protruding portions 3b and 3b. On the other hand, the second protruding portions 3c and 3c are required to be able to be easily removed when the bottom plate 3 is removed in a sliding manner from the housing body 2 with the gripper 3a being gripped. For this reason, the protruding amount of each of the second protruding portions 3c and 3c from the longer side of the bottom plate 3 is smaller than the first protruding portions 3b and 3b. In addition, the shape of each of the second protruding portions 3c and 3c is formed to have a curved section.

A cutout 3d having an elongated rectangular shape is formed in the other end portion, in the longitudinal direction, of the bottom plate 3. A dead end portion 3e of the cutout 3d is formed so that the position of the dead end portion 3e matches a part of the positioning hole 2e formed in the surface of the housing body 2 when the housing body 2 and the bottom plate 3 are assembled as shown in FIG. 1.

When the sensor chips SC housed in the chip package 1 are transferred to the chip transfer block 31, the positioning pin 34 of the chip transfer block 31 is inserted, as described above, from the bottom plate 3 side into the positioning hole 2e of the housing body 2 (at this time, the positioning pin 34 is brought into contact with the dead end portion 3e of the cutout 3d). Then, after the chip package 1 is positioned on the chip transfer block 31, the sensor chips SC are transferred. The cutout 3d is formed in order to avoid the interference of the positioning pin 34 with the bottom plate 3, which would otherwise prevent the bottom plate 3 from being pulled out of the housing body 2. The cutout 3d thus formed allows the positioning pin 34 to pass therethrough when the bottom plate 3 is pulled out of the housing body 2.

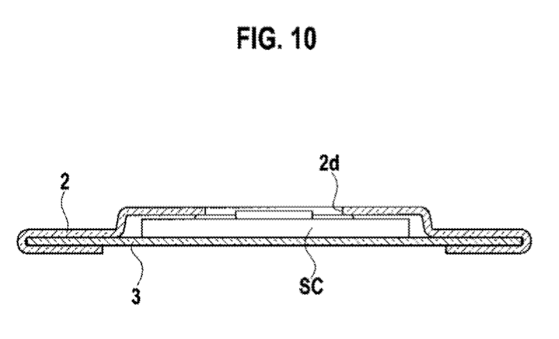

FIG. 9 is a plan view of the chip package 1, which shows a state where the sensor chips SC are housed in the chip package 1. The sensor chips SC are housed respectively in the pockets 2b, while the partition wall 2c is formed between each adjacent two of the sensor chips SC. FIG. 10 is a cross-sectional view showing the chip package 1 in which the sensor chips SC are housed as shown in FIG. 9, and taken along the line F-F. Each of the sensor chips SC is housed in the pocket 2b in a manner that the lower surface of the sensor chip SC is in contact with the bottom plate 3. In addition, the sample injection portion of each of the sensor chips SC does not protrude above the surface (the opening portion 2d) of the housing body 2, which surface corresponds to the upper surface of the pockets 2b. This is because of the following reason. In order to prevent dust or the like from entering the sample injection portion, a seal is attached to the opening portion 2d until just before a sample is injected into the sample injection portion. However, if the sample injection portions protrude above the surface of the housing body 2, the seal would be brought into contact with the projecting sample injection portions. Accordingly, the height of the sample injection portions is set as above so as to avoid such situation.

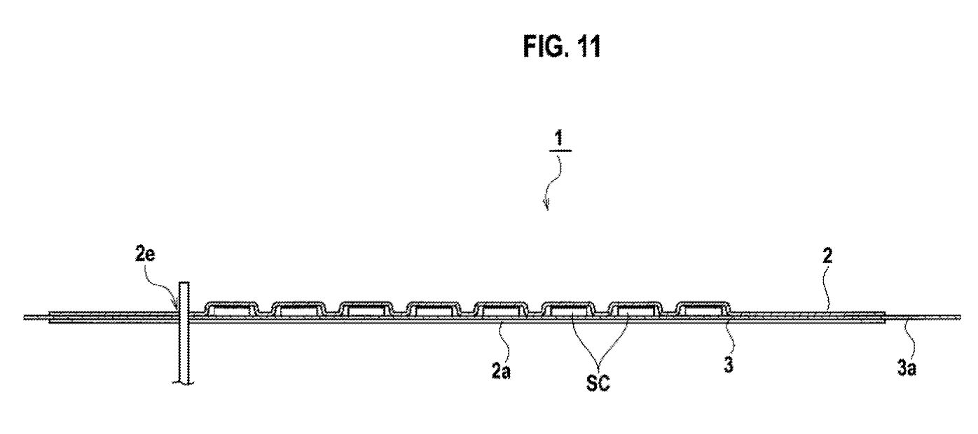

FIG. 11 is a cross-sectional view briefly showing, by cutting in the longitudinal direction, a state where the chip package 1 is fixed with the positioning pin 34 of the chip transfer block 31 inserted through the cutout 3d (the dead end portion 3e) of the bottom plate 3 and through the positioning hole 2e of the housing body 2. In the chip package 1, the sensor chips SC are housed, and held by the bottom plate 3.

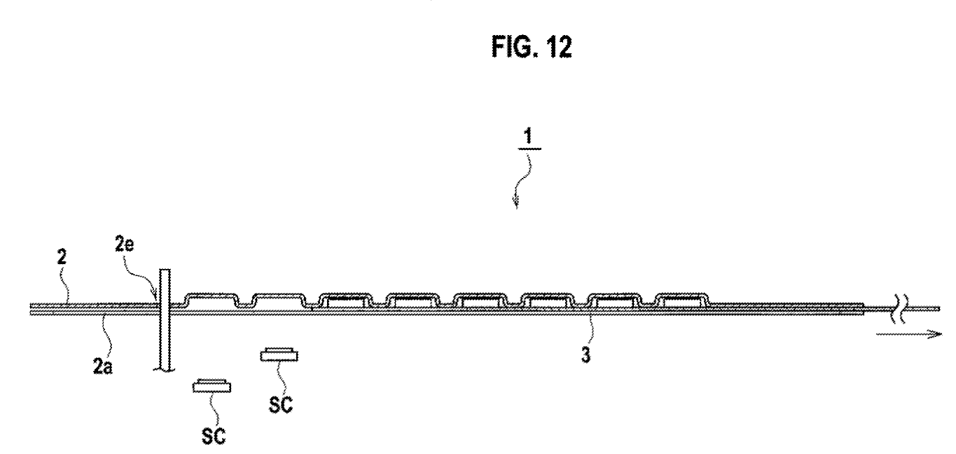

When the bottom plate 3 is pulled in a direction indicated by the arrow shown in FIG. 12 from such state, the bottom plate 3 moves on the folded-back portions 2a. As a result, the sensor chips SC, having been held by the bottom plate 3, fall down. Since the bottom plate 3 has the cutout 3d formed therein, the bottom plate 3 can be pulled off the housing body 2 without getting stuck by the positioning pin 34. Meanwhile, since the positioning pin 34 is inserted through the positioning hole 2e, the housing body 2 can maintain its fixed state. Accordingly, the multiple sensor chips SC housed in the chip package 1 can be transferred all together onto the chip transfer block 31.

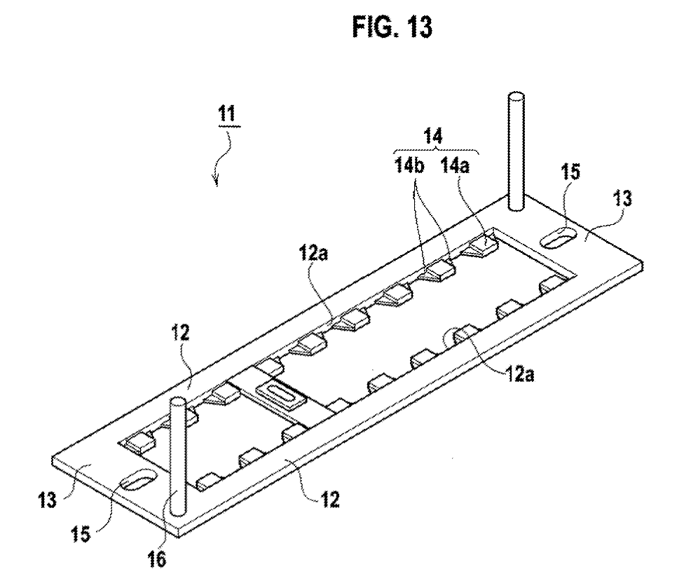

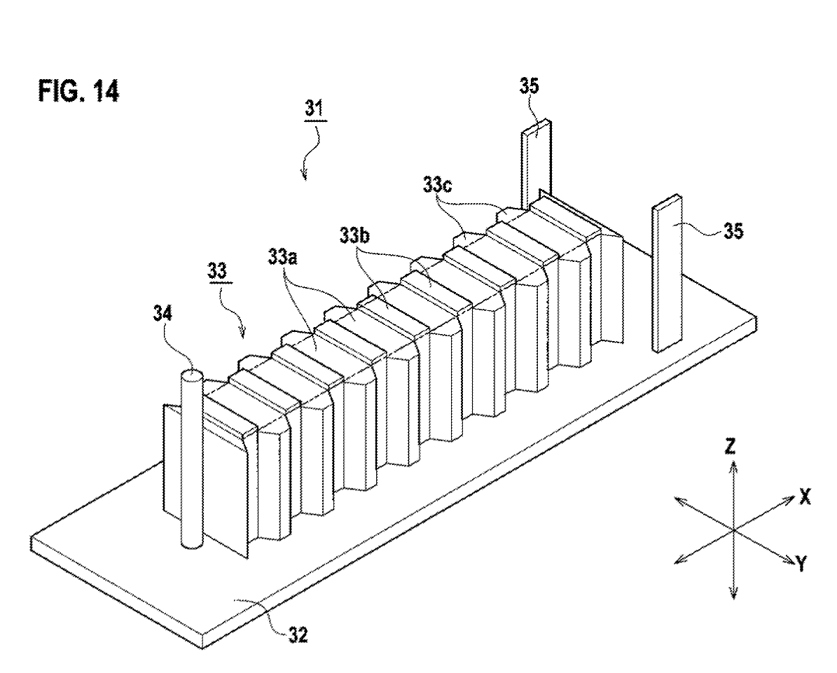

Next, the chip pallet 11 is used for transferring, to another place, the sensor chips SC having transferred onto the chip transfer block 31. As shown in FIG. 13, the chip pallet 11 is a rectangular-shaped pallet onto which the multiple sensor chips SC are transferred. Although, FIG. 13 shows a state where only one sensor chip SC has been transferred onto the chip pallet 11, the chip pallet 11 according to the embodiment of the present invention is configured so that eight sensor chips SC in total can be transferred onto the chip pallet 11.

The chip pallet 11 is formed of a pair of lateral frames 12 and 12 extending in parallel with each other; and a pair of vertical frames 13 and 13 extending in parallel with each other and connecting end portions of the lateral frames 12 and 12 on each side. Multiple positioning ribs 14 are formed at equal intervals on each of inside edge portions 12a and 12a, facing each other, of the respective lateral frames 12 and 12. In the chip pallet 11 according to the embodiment of the present invention, nine positioning ribs 14 are formed on each of the inside edge portions 12a and 12a, so that eight sensor chips SC can be mounted on the chip pallet 11.

Each of the positioning ribs 14 is formed of a first rib 14a which defines the arrangement positions of the sensor chips SC to be mounted on the chip pallet 11; and second ribs 14b onto which the sensor chip SC is transferred. Each of the first ribs 14a has the shape of a substantially rectangular parallelepiped. The sensor chip SC is placed between each two first ribs 14a and 14a adjacent to each other. Each of the second ribs 14b is provided in a substantially triangular shape as connecting the first rib 14a and the inside edge portion 12a. The sensor chip SC is mounted on the chip pallet 11 in a way that the four corners of the sensor chip SC is placed on the second ribs 14b, each two of which are provided between the adjacent two first ribs 14a and 14a on each side. In addition, since the second ribs 14b are formed to have a height lower than that of the first ribs 14a, the position at which each sensor chip SC is mounted on the chip pallet 11 is determined by the positioning ribs 14 and the inside edge portions 12a.

Holes 15 and 15 are formed in intermediate portions respectively of the pair of vertical frames 13 and 13. The positioning pin 34 provided on and protruding from the chip transfer block 31 is inserted through one of the holes 15 and 15. In addition, transport rods 16 for transporting the chip pallet 11 are provided respectively on parts each connecting the lateral frame 12 and the vertical frame 13. In the chip pallet 11, a pair of the transport rods 16 are provided at positions opposite to each other.

FIG. 14 is a perspective view showing the chip transfer block 31, which is used for transferring the sensor chips SC from the chip package 1 onto the chip pallet 11. The chip transfer block 31 includes: a base 32, which has a rectangular shape in the embodiment of the present invention; a transfer stage 33 provided on the base 32; the positioning pin 34; and a pair of positioning guides 35 and 35.

The transfer stage 33 is provided on the base 32, and the sensor chips SC housed in the chip package 1 are transferred onto the transfer stage 33. Then, the sensor chips SC are transferred from the transfer stage 33 onto the chip pallet 11. The transfer stage 33 is formed, on the base 32, so as to have a certain thickness in the Z-axis direction shown in FIG. 14. This thickness of the transfer stage 33 is sufficient as long as being, at least, equal to or more than the thickness of the chip pallet 11 to be transferred.

Faces 33a and bars 33b are formed on a surface, opposite to the surface connected to the base 32, of the transfer stage 33. The sensor chips SC transferred from the chip package 1 are mounted on the faces 33a (hereinafter, referred to as “mounting faces”). The bars 33b are formed each between adjacent two of the mounting faces 33a.

The bars 33b are formed to have a height, in the Z-axis direction, greater than that of the mounting faces 33a. In addition, the bars 33b extend in a direction parallel to the longitudinal direction (the Y-axis direction in FIG. 14) of the sensor chips SC to be mounted on the mounting faces 33a. The formation of the bar 33b between the adjacent two mounting faces 33a and 33a makes it possible to prevent the sensor chip SC from being displaced from the mounting face 33a. The distance between each adjacent two of the bars 33b is substantially the same as the length of each sensor chip SC in a direction along the shorter sides of the sensor chip SC. The sensor chips SC are mounted respectively on the mounting faces 33a each formed to have that length.

The length, in the Y-axis direction, of each mounting face 33a is shorter than the length, in the longitudinal direction, of each sensor chip SC. For this reason, end portions, in the longitudinal direction, of each sensor chip SC protrude from the mounting face 33a. The mounting faces 33a have such structure for the following reason. If the mounting faces 33a were formed so that the entire region of the lower surface of the sensor chip SC is mounted on the mounting face 33a, the sensor chips SC could not be transferred to the chip pallet 11 by use of the chip transfer block 31.

Convex portions 33c are formed on surfaces of the transfer stage 33, which are orthogonal to the mounting faces 33a and also orthogonal to the longitudinal direction of the sensor chips SC. Specifically, each convex portion 33c is formed to protrude to the outer side of the transfer stage 33, in a region directly below one of the two end portions of the sensor chip SC, between the mounting face 33a and the base 32. In the embodiment of the present invention shown in FIG. 14, the convex portions 33c are formed to have such a height from the base 32 as to form horizontal surfaces along with the mounting faces 33a. However, the height of the convex portions 33c may be set as desired, as long as insertion and removal operations carried out between the transfer stage 33 and the chip pallet 11, which will be described later, can be smoothly performed.

In the embodiment of the present invention, each convex portion 33c has, as shown in FIG. 14, a substantially trapezoidal shape having a bottom line on the side where the convex portion 33c is connected to the mounting face 33a. This shape of the convex portion 33c is designed 80 as to be able to be fitted on the inside edge portion of the chip pallet 11 when the chip pallet 11 is attached to the chip transfer block 31. Specifically, the chip pallet 11 is fitted to the chip transfer block 31 in a manner that each of the convex portions 33c of the transfer stage 33 is placed between the corresponding two positioning ribs 14 and 14 of the chip pallet 11. In addition, since each convex portion 33c is formed in such shape, the four corners of the lower surface of each sensor chip SC are not held by the convex portion 33c. When the chip pallet 11, which has been fitted in advance to the transfer stage 33, is pulled up, each sensor chip SC is stuck, at the four corners thereof, by the second ribs 14b of the chip pallet 11, thus being mounted on the second ribs 14b. As a result, the sensor chips SC are transferred onto the chip pallet 11.

The positioning pin 34 is provided on a portion in the vicinity of the center of one end portion, in the longitudinal direction (the X-axis direction) of the transfer stage 33. The positioning pin 34 is used for positioning the chip package 1 when the sensor chips SC are to be transferred onto the transfer stage 33. The position, in the longitudinal direction, of the chip package 1 is set on the transfer stage 33 in a way that the positioning pin 34 is inserted through the cutout 3d of the bottom plate 3 and through the positioning hole 2e of the housing body 2.

In addition, the positioning guides 85 aligned with the Y-axis direction of the transfer stage 33 are formed on portions in the vicinity of the other end portion, in the longitudinal direction, of the transfer stage 33. These positioning guides 35 are used for positioning the chip package 1 in the direction along the shorter sides of the chip package 1. These positioning guides 35 are formed on portions in the vicinity of the other end portion, in the longitudinal direction of the transfer stage 33 in the embodiment of the present invention. However, these positioning guides 35 may be formed on any portions, in the longitudinal direction, of the transfer stage 33 as long as these positioning guides 35 allow the chip package 1 to be positioned in the direction along the shorter sides of the chip package 1. Moreover, although formed at positions opposite to each other in FIG. 14, the positioning guides 35 do not necessary need to be formed at positions opposite to each other. Alternatively, the positioning guides 35 may be formed, while displaced with respect to each other, respectively on the two sides with the transfer stage 33 sandwiched in between.

Next, a part of a method for measuring a sample housed in the sensor chips SC will be described. The part to be described here is a procedure that the sensor chips SC are transferred onto the chip pallet 1 after the sensor chips SC housed in the chip package 1 are once transferred onto the chip transfer block 31.

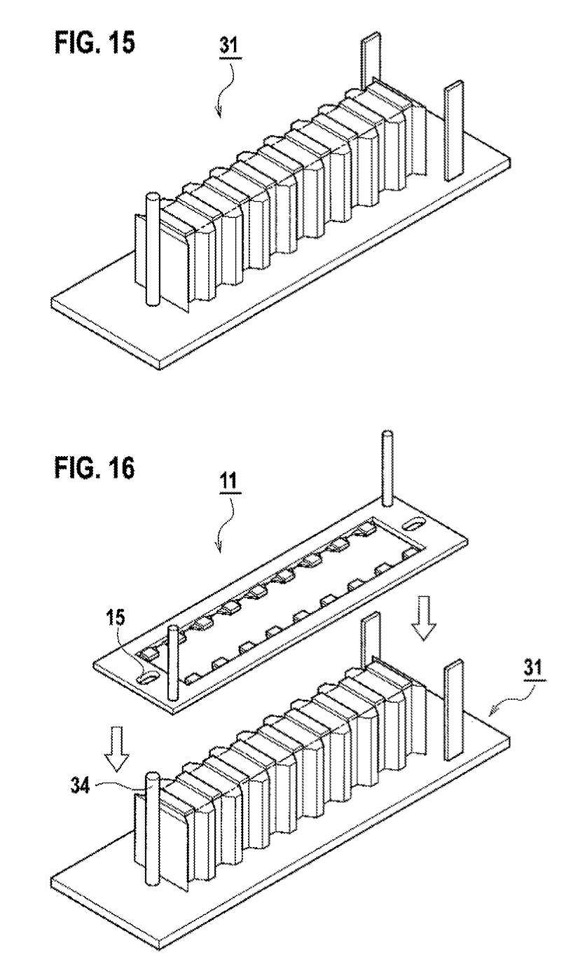

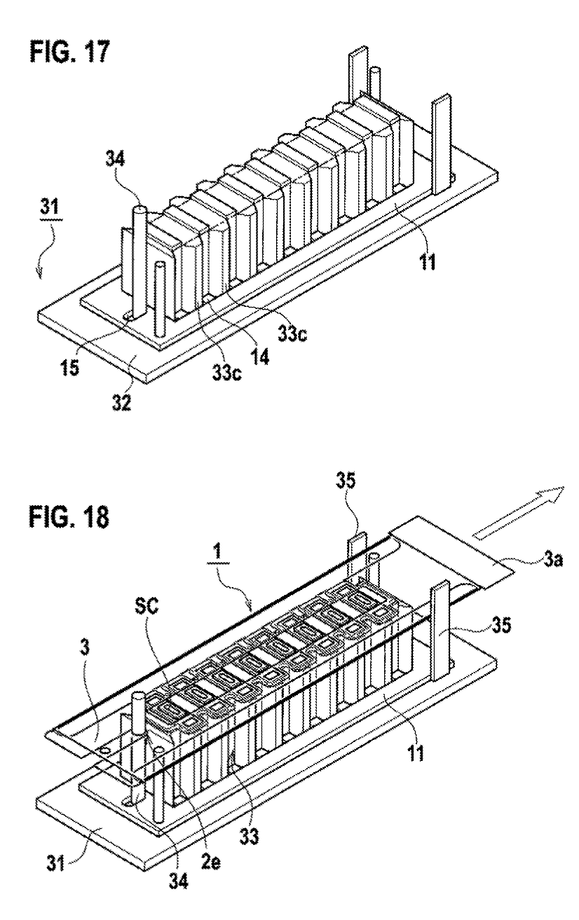

As shown in FIG. 15, first of all, the chip transfer block 31 is prepared. Next, the chip pallet 11 for transporting the sensor chips SC to a measuring device not illustrated here is fitted onto the transfer stage 33 of the chip transfer block 31 (refer to FIG. 16). Having a frame-like shape as shown in FIG. 13, the chip pallet 11 is fitted in a manner that the transfer stage 33 is inserted through the inside of the frame-like shape as indicated by the arrow in FIG. 16. FIG. 17 shows a state where the chip pallet 11 is mounted on the base 32 of the chip transfer block 31. As shown in FIG. 17, the positioning ribs 14 enter each between the corresponding two convex portions 33c and 33c of the transfer stage 33. In addition, the positioning pin 34 of the chip transfer block 31 is inserted through the hole 15 formed in the chip pallet 11.

FIG. 18 is a process view showing a state where the positioning pin 34 is inserted further through the positioning hole 2e of the chip package 1 while the chip pallet 11 has been mounted on the base 32 of the chip transfer block 31. In FIG. 18, the chip package 1 is drawn transparently, so that the positional relationship between the chip transfer block 31 and the chip pallet 11 is clearly shown.

In this state, the two edges of the chip package 1 in the direction along the shorter sides thereof, are sandwiched by the pair of positioning guides 35 formed on the chip transfer block 31. The position of the chip package 1 is thus set on the transfer stage 33. In addition, in this state, the bottom plate 3 of the chip package 1 is placed on the bars 33b of the transfer stage 33. In the state where the chip package 1 is placed at such position on the transfer stage 33, the sensor chips SC housed in the chip package 1 are located respectively above the mounting faces 33a.

Now, pulling the gripper 3a of the bottom plate 3 in the chip package 1 in a direction indicated by the arrow shown in FIG. 18 causes the sensor chips SC housed in the chip package 1 to fall down onto the mounting faces 33a on the transfer stage 33.

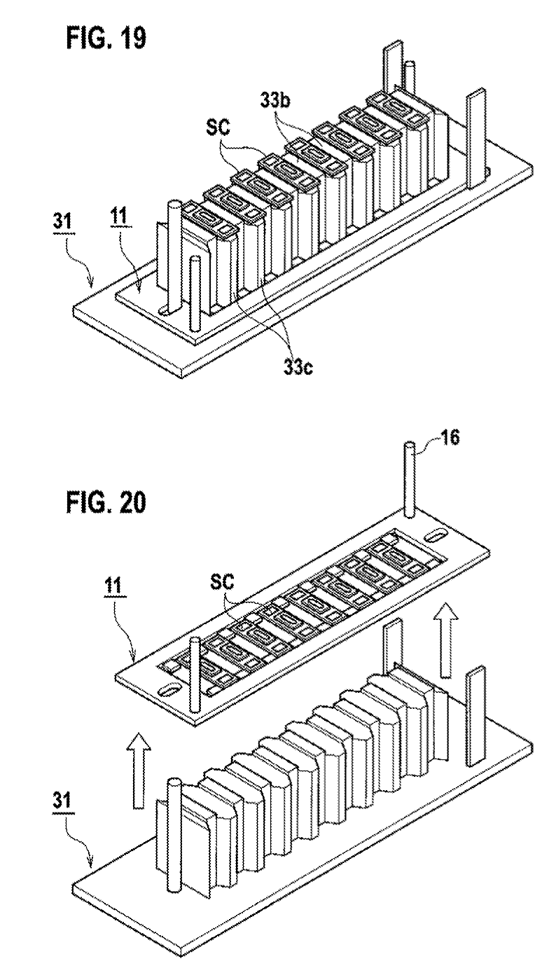

FIG. 19 is a process view showing a state where the sensor chips SC have been transferred from the chip package 1 onto the transfer stage 33 of the chip transfer block 31. In FIG. 19, the chip package 1 from which the sensor chips SC housed therein have been transferred onto the transfer stage 33 is not illustrated. Each sensor chip SC having fallen down from the chip package 1 and been mounted on the mounting face 33a is sandwiched by the bars 33b and 33b, and the two end portions, in the longitudinal direction, of the sensor chip SC are placed respectively on the convex portions 33c. Meanwhile, the four corners of the sensor chip SC are not placed on the convex portions 33c, and are not held by the convex portions 33c.

In this state, the chip pallet 11 having been fitted onto the chip transfer block 31 is pulled up. FIG. 20 shows how the chip pallet 11 is pulled up. The chip pallet 11 is pulled up, with the transport rods 16 being gripped, along the convex portions 33c and the positioning pin 34 in a direction indicated by the arrow shown in FIG. 20. In this event, the four corners, not having been held, of each sensor chip SC are mounted on the second ribs 14b of the chip pallet 11. Then, the chip pallet 11 is further pulled up and removed from the chip transfer block 31. Thereby, the sensor chips SC are transferred onto the chip pallet 11.

Then, the chip pallet 11 having the multiple sensor chips SC mounted thereon is placed on the unillustrated measuring device, and the sample in the sensor chips SC is measured.

Using the chip package 1, the chip pallet 11, and the chip transfer block 31, which have been described so far, makes it possible to provide a method for measuring a sample, a chip package for sensor chips, and a mechanism for fixing sensor chips, all of which enable the operator to easily, promptly, and also safely, transfer multiple sensor chips onto a measuring device indirectly, or without using any tool, and to fix the sensor chips with a simple structure, when transferring the sensor chips onto the measuring device in order to measure samples in the sensor chips.

### Second Embodiment

Next, a second embodiment of the present invention will be described. Note that, in the second embodiment, the same constituent elements as those described in the above first embodiment are denoted by the same reference numerals, and the overlapping description thereof will be omitted.

The second embodiment shows procedures of transferring, mounting, and then fixing sensor chips onto a measuring device in the method of measuring samples.

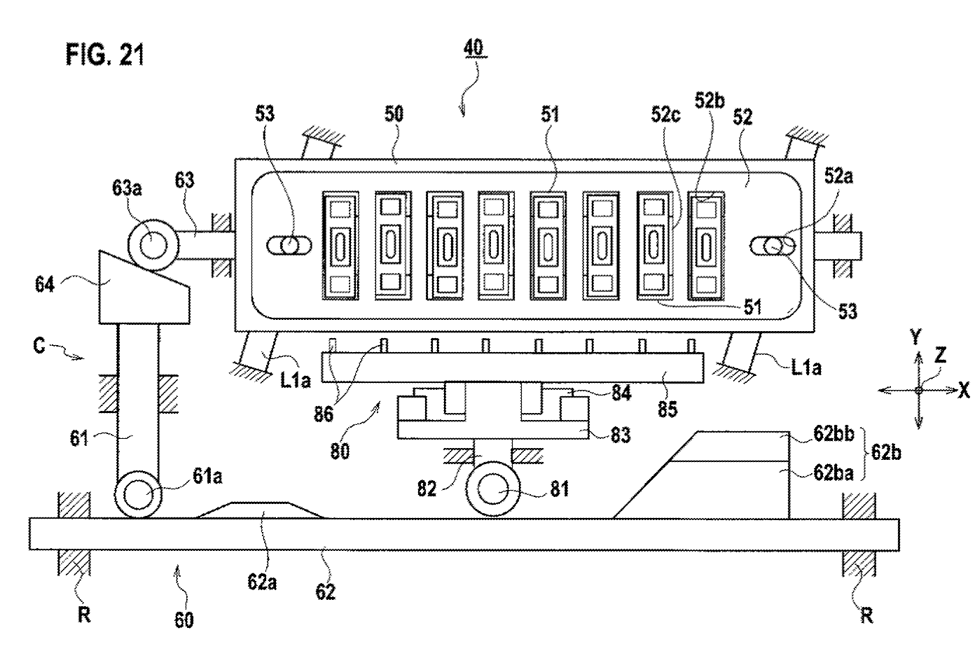

FIG. 21 is a schematic view showing a fixing device for fixing sensor chips on a measuring device for reading samples in the sensor chips.

A fixing device 40 includes a chip-fixing block 50, a displacement mechanism 60, and a fixing jig 80. Sensor chips SC are mounted on the chip-fixing block 50. The displacement mechanism 60 is configured to move the chip-fixing block 50, thereby displacing the sensor chips SC to positions where the sensor chips SC are to be fixed, respectively. The fixing jig 80 fixes the position of the sensor chips SC displaced on the chip-fixing block 50. All of the chip-fixing block 50, the displacement mechanism 60, and the fixing jig 80 are provided to a measuring device R, which is not illustrated in FIG. 21.

The sensor chips SC are mounted on the chip-fixing block 50, and fixed thereto by the fixing jig 80. Then, the samples contained in the insides of the sensor chips SC are read by a reading device (a scanning mechanism) provided to the measuring device R.

In the chip-fixing block 50, guides 51 for mounting the sensor chips SC are formed. Each of the guides 51 is formed to be capable of three sides, including a shorter side, of the sensor chip SC. In addition, each pair of guides 51 and 51 are formed at such positions that parts, each of which holds the shorter side, of the respective guides 51 and 51 face each other. Each pair of these guides 51 and 51 hold one sensor chip SC. Multiple pairs of guides 51 and 51 (eight pairs in the embodiment of the present invention) are formed along the longitudinal direction indicated by the Y-axis direction in FIG. 21.

Moreover, the chip-fixing block 50 is provided with positioning pins 53 for positioning the chip-discharging pallet 52 to be mounted on the chip-fixing block 50. In the embodiment of the present invention, a pair of the positioning pins 53 are provided, each at the center in the width direction (the X-axis direction) of the chip-fixing block 50, in the manner of sandwiching the multiple guides 51.

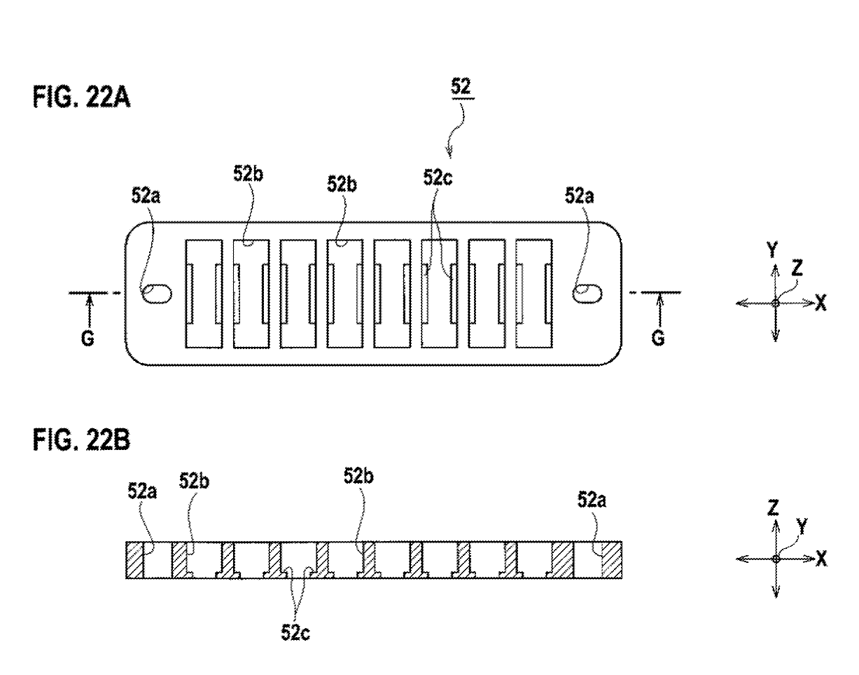

The chip-discharging pallet 52 is a pallet for discharging the sensor chips SC from the measuring device R after the sensor chips SC are mounted on the chip-fixing block 50 and then the samples are read by the measuring device R. FIG. 22A is a plan view showing the chip-discharging pallet 52, and FIG. 22B is a cross-sectional view showing the chip-discharging pallet 52 taken along the line G-G in FIG. 22A. As shown in FIG. 22A, the chip-discharging pallet 52 is provided positioning holes 52a and 52a formed therein. The chip-discharging pallet 52 is positioned with the positioning pins 53 and 53 inserted respectively through the positioning holes 52a and 52a when the chip-discharging pallet 52 is mounted on the chip-fixing block 50. Although both of the positioning holes 52a and 52a are formed in elongated holes in the embodiment of the present invention, it is sufficient that at least one of these positioning holes 52a and 52a is formed in an elongated hole.

Mounting pockets 52b for housing the sensor chips SC are formed in the chip-discharging pallet 62 in a manner that the mounting pockets 52b are sandwiched by the positioning holes 52a and 52a. In the embodiment of the present invention, eight mounting pockets 52b are formed. In addition, a pair of mounting pieces 52c and 52c are formed respectively on two sides, facing each other in the X-axis direction, of each of the mounting pockets 52b. When the sensor chips SC housed respectively in the mounting pockets 52b mounted on the guides 51 are discharged, the lower surface of each of the sensor chips SC is brought into contact with the mounting pieces 52c and 52c. Accordingly, when the chip-discharging pallet 52 is pulled off the chip-firing block 50, the sensor chips SC are also pulled off the chip-fixing block 50 at the same time.

As shown in FIG. 21, when the chip-discharging pallet 52 is mounted on the chip-fixing block 50, the guides 51 formed on the chip-fixing block 50 are housed respectively in the mounting pockets 52b. Thus, in a state where the chip-discharging pallet 52 is mounted on the chip-fixing block 50 and where the sensor chips SC are thus housed respectively in the mounting pockets 52b, the lower surfaces of the sensor chips SC are in contact with the guides 51. However, each of the mounting pockets 52b is formed to have a size larger than that of each of the sensor chips SC. For this reason, in a state where the sensor chips SC are just housed in the mounting pockets 52b, the positions of the respective sensor chips SC are not uniform in the corresponding mounting pockets 52b as shown in FIG. 21.

The displacement mechanism 60 includes, as shown in FIG. 21, a cam mechanism C and a pair of fret liner guide bearings L1 and L1. The cam mechanism C includes a first cam 62 and a second cam 64. The first com 62 moves a first follower 61 in one direction. The second com 64 is connected to the first follower 61, and moves a second follower 63, which is connected to the chip-fixing block 50, in a direction orthogonal to the direction in which the first follower 61 moves. In addition, the pair of first linear guide bearings L1 and L1 are connected to the second follower 63, and move the chip fixing block 50 in a direction not orthogonal to the direction in which the second follower 63 moves. The displacement mechanism 60 including these components is movably provided on the measuring device R.

The first cam 62 is a translate cam as shown in FIG. 21. A first bump 62a and a second bump 62b are formed in the first cam 62. The first bump 62a is used for aligning the sensor chips SC. The second bump 62b is used for fixing the sensor chips SC thus aligned. The second bump 62b is formed to be higher than the first bump 62a in the Y-axis direction, and is also formed to have two heights of a lower second bump 62ba and a higher second bump 62bb. The lower second bump 62ba is designed to push out the fixing jig 80 in the Y-axis direction above the sensor chips SC. The higher second bump 62bb is designed to move, in the Z-axis direction, the fixing jig 80 having reached above the sensor chips SC for fixing the sensor chips SC, so as to fix the sensor chips SC.

The first follower 61 has the cam follower 61a and the second cam 64. The cam follower 61a is provide to one end portion of the first follower 61, the one end portion being in contact with the first cam 62. The second cam 64 is formed on the other end portion of the first follower 61. The cam follower 61a is designed to be capable of moving on the first cam 62. The second cam 64 is a cam for moving the second follower 63, and is formed in a substantially trapezoidal shape which has a slop inclined downward while extending toward the chip-fixing block 50 in the X-axis direction Having such shape, the second cam 64 is capable of moving the second follower 63 to the right in the X-axis direction shown in FIG. 21 at the beginning.

What is moved by the second cam 64 is the second follower 63. One end portion of the second follower 63 has the cam follower 63a attached thereto. The other end portion of the second follower 63 is formed into a free end which is allowed to freely move in the X-axis direction.

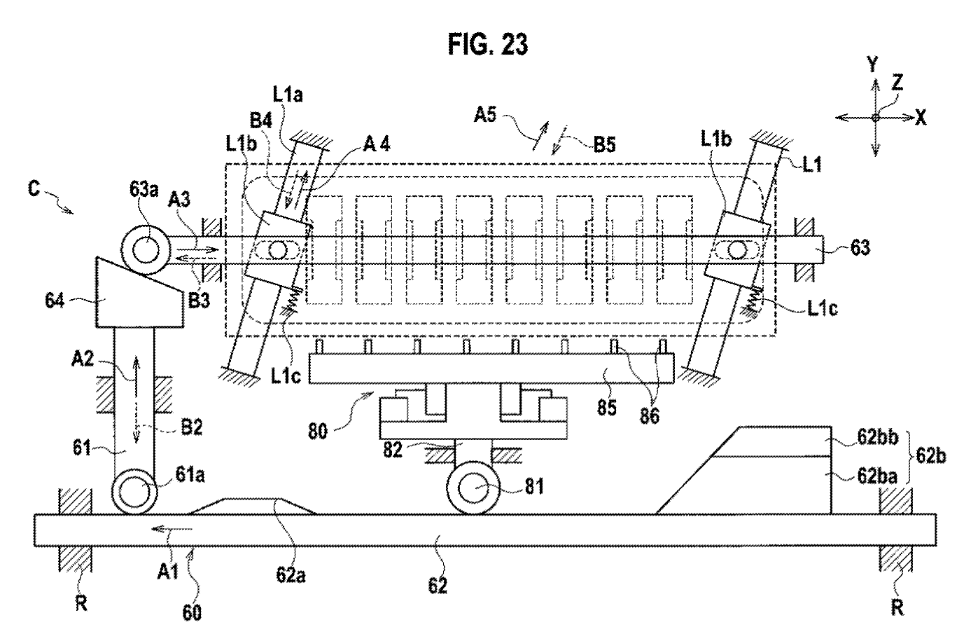

FIG. 23 is a schematic view showing a relationship between the cam mechanism C, which is provided on the lower surface of the chip-fixing block 50, and the first linear guide bearings L1, and more specifically, showing a relationship between the second follower 63 and the first linear guide bearings L1. In FIG. 23, the chip-fixing block 50 and the chip-discharging pallet 52 mounted thereon are indicated by the dashed lines.

The second follower 63 is provided in the manner of penetrating the chip-fixing block 50 in the X-axis direction, and is connected to the first linear guide bearings L1 and L1 respectively at two positions below the second follower 63 in the Z-axis direction. The second follower 63 and each first linear bearing L1 are coupled to each other by a pin. This pin is also connected to the chip-fixing block 50, and further, is utilized as the positioning pin 53 for positioning the chip-discharging pallet 52 on the chip-fixing block 50.

Each of the first linear guide bearings L1 includes a rail member L1a and a bearing member L1b which moves on and along the rail member L1a. Each first linear guide bearing L1 is fixed to the measuring device R so as to extend in a direction which is not orthogonal to the second follower 63 (a non-orthogonal direction). In addition, an urging member L1c is connected to each bearing member L1b.

Hereinafter, description will be given of how the chip-fixing block 60 is moved by the cam mechanism C and the first linear guide bearings L1 and L1 all included in the displacement mechanism 60. Specifically, moving the first cam 62 in the X-axis direction (the direction indicated by the solid arrow A1) causes the cam follower 61a to run onto the first bump 62a. When the cam follower 61a runs on the first bump 62a, the first follower 61 moves in the Y-axis direction (the direction indicated by the solid arrow A2) in the manner of pushing upward the second cam 64. Since the second cam 64 has such shape as described above, the second follower 63 is caused to move in the X-direction (the direction indicated by the solid arrow A3) in conjunction with the cam follower 63a by the second cam 64. Although the second follower 63 and the first cam 62 move respectively in the directions indicated by the arrows A1 and A3, that is, both in the X-axis direction, the directions of the movements thereof are opposite to each other.

When the second follower 63 moves in the direction of the arrow A3, the bearing member L1b of each first linear guide bearing L1 connected to the second follower 63 moves along the rail member L1a in the direction indicated by the arrow A4. In this event, the urging member L1c connected to the bearing member L1b extends in association with the movement of the bearing member Lib. The movement in the direction of the arrow A3 and the movement in the direction of the arrow A4 cause the chip-fixing block 50 to move in the direction indicated by the arrow A5.

When the first cam 62 further moves in the direction of the arrow A1, the cam follower 61a runs down from the first bump 62a. The first follower 61 then moves in the direction indicated by the dashed arrow B2, while the second follower 63 moves in the direction indicated by the dashed arrow B3. Moreover, in association with this movement of the second follower 63, the urging member L1c return to its original state, so that the bearing member Lib moves in the direction indicated by the dashed arrow B4. Accordingly, the chip fig block 50 moves in the direction indicated by the dashed arrow B5, and the first cam 62 returns to the state before the movements.

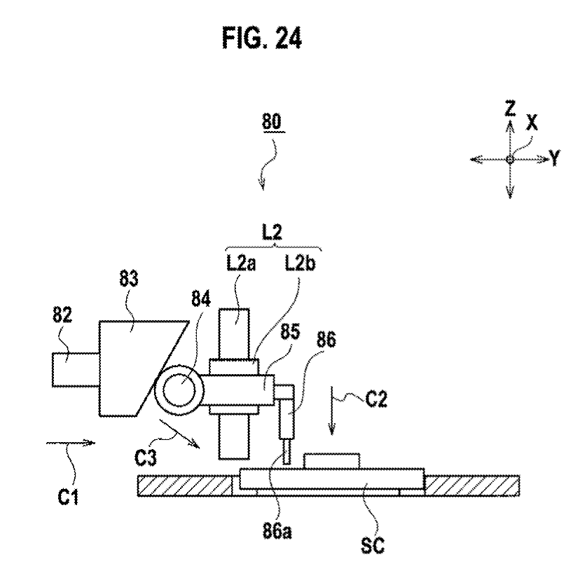

The fixing jig 80 includes, as shown in FIGS. 21 and 24, a cam follower 81, a third follower 82, a third cam 83, a cam follower 84, a fourth follower 85, a second linear guide bearing L2, and chip-fixing pins 86. The cam follower 81 is in contact with the first cam 62. The cam follower 81 is attached to one end of the third follower 82. The third cam 83 is provided on the other end of the third follower 82. The cam follower 84 is in contact with the third cam 83. The cam follower 84 is attached to one end of the fourth follower 85. The second linear guide bearing L2 moves the fourth follower 85 in the Z-axis direction. The chip-fixing pins 86 are connected to the other end of the fourth follower 85, and are brought into contact with, and thereby fix, the sensor chips SC, respectively.

Although not shown in FIG. 21, the cam follower 81 is further divided into a first cam follower 81a and a second cam follower 81b. This is because the first cam follower 81a and the second cam follower 81b play different roles from each other. The first cam follower 81a is designed to run onto the lower second bump 62ba so as to push and move the fixing jig 80 to a position above the chip-fixing block 50. On the other hand, the second cam follower 81b is designed as follows. The second cam follower 81 keeps climbing the second bump 62b even after the first cam follower 81a runs onto the lower second bump 62ba. Then, by running further onto the higher second bump 62bb, the second cam follower 81b moves, in the Z-a direction, the fixing jig 80 having been moved above the chip-fixing block 50. In this way, the second cam follower 81b fixes the sensor chips SC by use of the fixing jig 80.

FIG. 24 is a schematic view showing the fixing jig 80 having reached the position above the sensor chips SC as viewed in the X-axis direction. As shown in FIG. 24, the third cam 83 is formed in a substantially trapezoidal shape having a slope inclined downward while extending toward the chip-fixing block 50 so as to be capable of pushing down the fourth follower 85.

In addition, the fourth follower 85 having the cam follower 84 attached to the one end thereof is connected further to the second linear bearing L2. The second linear guide bearing L2 includes a rail member L2a and a bearing member L2b which moves on along the rail member L2a. Connected to the second linear guide bearing L2, the fourth follower 85 is capable of moving in the Z-axis direction.

As shown in FIG. 24, each chip-fixing pin 86 is attached to the other end of the fourth follower 85 in a manner that the chip-fixing pin 86 extends in a direction orthogonal to the fourth follower 85 and the Z-axis direction. Each chip-fixing pin 86 further includes a chip-fixing-pin contact probe 86a which is actually brought into contact with the sensor chips SC. An urging member is attached to one end, which is not brought into contact with the sensor chip SC, of the chip-fixing-pin contact probe 86a. This urging member absorbs the movement of the fourth follower 85 in association with the second linear guide bearing L2 when the chip-fixing-pin contact probe 86a is brought into contact with the sensor chip SC. In addition, the urging member allows the chip-fixing-pin contact probe 86a to be securely brought into contact with, and fixes, the sensor chip SC.

Specifically, as indicated by the arrow C1 in FIG. 24, the third follower 82 is pushed by the second cam follower 81b in the Y-axis direction. The third cam 83 has such shape as described above, and the fourth follower 85 is connected to the second linear guide bearing L2, and thus is movable in the Z-axis direction. Accordingly, the fourth follower 85 moves in the direction indicated by the arrow C2. Then, the movement in the direction of the arrow C1 and the movement in the direction of the arrow C2 cause the fourth follower 85 to move in the direction indicated by the arrow C3. Since the fourth follower 85 moves as above, the chip-fixing-pin contact probe 86a is pushed downward in the Z-axis direction, and is brought into contact with the surface of the sensor chip SC, thus Sing the sensor chip SC.

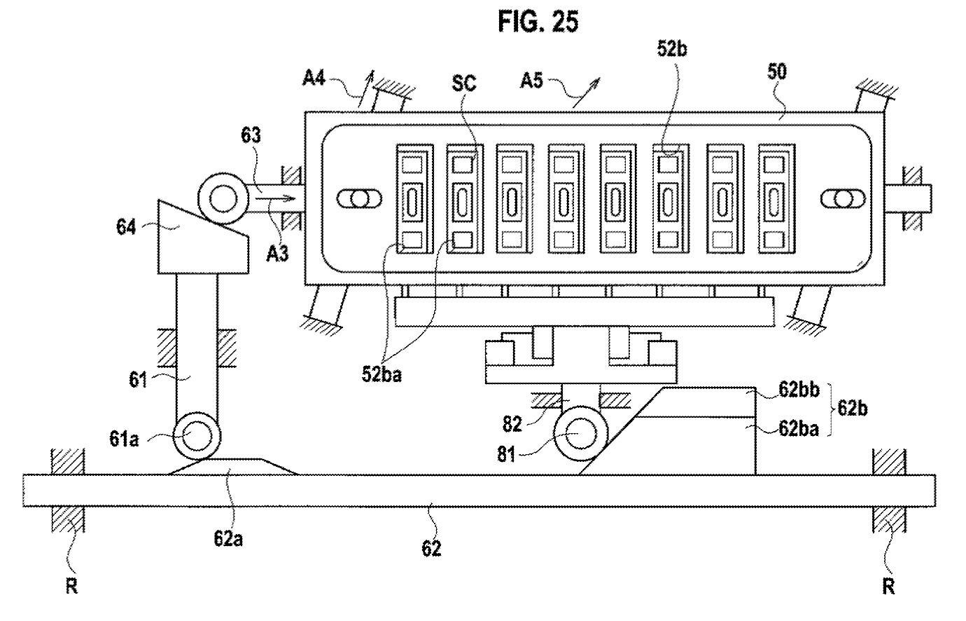

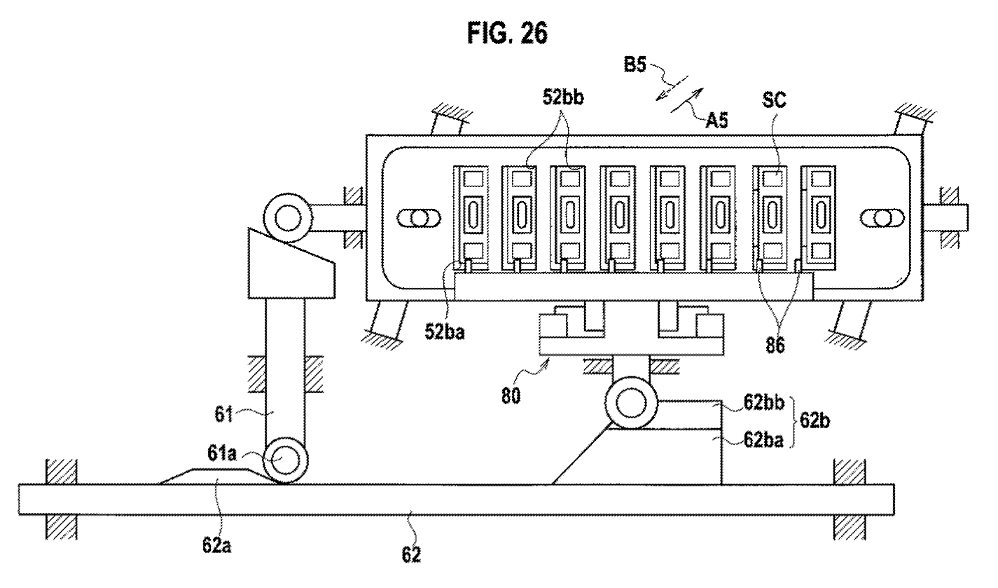

FIGS. 25 and 26 are schematic views for explaining how the sensor chips SC are moved and fixed by the movement of the displacement mechanism 60. The positions of the sensor chips SC are not aligned, as shown in FIG. 21, immediately after the sensor chips SC are transferred from the chip package 1, and housed in the mounting pockets 52b of the chip-discharging pallet 52.

FIG. 25 shows a state where the cam follower 61a (the first follower 61) has run onto the first bump 62a. In this state, as described by referring to FIG. 23, the second follower 63 is moved by the second cam 64 in the X-axis direction, and in the direction (the direction of the arrow A3) opposite to that in which the first cam 62 has moved. The chip-fixing block 50 moves in the direction of the arrow A5 in association with the movement of the first linear guide bearings L1 and L1 in the direction of the arrow A4. The mounting pockets 52b are formed to be larger than the sensor chips SC which are housed respectively in the mounting pockets 52b. For this reason, if the movement of the chip-fixing block 50 is sharp, the sensor chips SC do not follow the movement of the chip-fixing block 50. As a result, each of the sensor chips SC hits a first corner 52ba of the mounting pocket 52b, and thus, is stopped.

The cam follower 81 (the third follower 82) also runs onto the second bump 62b at the same time when the cam follower 61a (the first follower 61) runs onto the first bump 62a. Such movement of the cam follower 81 pushes the first jig 80 from the position shown in FIG. 21 toward the chip-fixing block 50.

As shown in FIG. 26, once the cam follower 61a (the first follower 61) rums down from the first bump 62a, the chip-fixing block 50 having been moved in the direction of the arrow A5 is further moved in the direction opposite to that of the arrow A5 (the direction indicated by the arrow B5). The sensor chips SC do not follow this movement of the chip-fixing block 50 as well. Accordingly, at this time, each of the sensor chips SC hits a second corner 52bb diagonally opposite to the first corner 52ba, and thus, is stopped. When the sensor chips SC are brought into such state, the chip-fixing-pin contact probes 86a of the fixing jig 80 are also moved to positions where the chip-fixing-pin contact probes 86a can be brought into contact with the sensor chips SC. The second cam follower 81b then runs onto the higher second bump 62bb, so that the sensor chips SC are fixed by the chip-fixing pin contact probes 86a.

When the sensor chips SC are transferred from the chip package 1, the positions of the sensor chips SC in the mounting pockets 52b are not aligned. The above-described operation of the displacement mechanism 60 eventually causes each of the sensor chips SC to come into contact with a specific corner (the second corner 52bb in the embodiment of the present invention) of the mounting pocket 52b, and also to be fixed by the fixing jig 80.

Using the chip package 1, the chip-fixing block 50, the chip-discharging pallet 52, the displacement mechanism 60, and the fixing jig 80, which have been described so f makes it possible to provide a method for measuring a sample, a chip package for sensor chips, and a mechanism for fixing sensor chips, all of which enable the operator to easily, promptly, and also safely, transfer multiple sensor chips onto a measuring device indirectly, or without using any tool, and to fix the sensor chips with a simple structure, when transferring the sensor chips onto the measuring device in order to measure samples in the sensor chips.

It should be noted that, the present invention is not limited to the above-described embodiments as they are, and may be embodied, when to be implemented, with modification made on the constituent elements without departing from the scope of the present invention. Moreover, a variety of inventions may be formed by combining, as appropriate, multiple constituent elements out of those disclosed in A the above-described embodiments. For example, some constituent elements may be omitted from among all the constituent elements shown in the embodiments. Furthermore, constituent elements of the different embodiments may be appropriately combined as appropriate.

What is claimed is:

1. A method for measuring a sample, comprising;
fitting and mounting, onto a transfer stage, a frame-shaped chip pallet for transporting a plurality of sensor chips, the transfer stage being provided in a chip transfer block;
fitting a positioning hole of a chip package having the sensor chips housed therein, on a positioning pin provided on the chip transfer block, so as to position the chip package, in a longitudinal direction, on the transfer stage;
pulling, off the chip package, a bottom plate thereof which holds lower surfaces of the sensor chips, so as to simultaneously transfer the sensor chips respectively onto positions each between bars for preventing the sensor chips from moving, the bars being formed on a mounting face, onto which the sensor chips are mounted, of the transfer stage;
pulling upward the chip pallet to bring the chip pallet into contact with four corners of each of the sensor chips mounted on the transfer stage, so that the sensor chips are simultaneously transferred onto the chip pallet; and
mounting the chip pallet having the sensor chips mounted thereon onto a measuring device, and measuring a sample in the sensor chips.

2. A chip package comprising:
a housing body having a plurality of chip pockets formed at equal intervals, the chip pockets respectively housing a plurality of sensor chips each having a sample injection portion formed in an upper surface thereof, the chip pockets having an opening portion exposing the sample injection portions to the outside; and
a bottom plate for holding lower surfaces of the respective sensor chips, wherein
the bottom plate and the housing body are assembled in a manner that the bottom plate is slidable along and between folded-back portions formed in the housing.

3. The chip package according to claim 2, further comprising a seal member which is attached to the opening portion so as to cover the opening portion.

4. The chip package according to claim 2, wherein:
a positioning hole is formed in the housing,
when the sensor chips are mounted on a transfer stage provided in a chip transfer block, a positioning pin provided on the chip transfer block is inserted through the positioning hole for positioning the sensor chips on the transfer stage, and
a cutout is formed in the bottom plate, so that the positioning pin passes through the cutout when the bottom plate is pulled off the housing.

5. The chip package according to claim 3, wherein:
a positioning hole is formed in the housing,
when the sensor chips are mounted on a transfer stage provided in a chip transfer block, a positioning pin provided on the chip transfer block is inserted through the positioning hole for positioning the sensor chips on the transfer stage, and
the bottom plate is provided with a cutout through which the positioning pin passes when the bottom plate is pulled off the housing.

6. A method for measuring a sample, comprising:
mounting a chip-discharging pallet onto a chip-fixing block of a measuring device for reading samples in a plurality of sensor chips;
fitting a positioning hole of a chip package, which houses the sensor chips, onto a positioning pin provided on the chip-fixing block, so as to set, on the transfer stage, a position, in a longitudinal direction, of the chip package;
pulling a bottom plate, which holds lower surfaces of the sensor chips, off the chip package, so as to simultaneously transfer the sensor chips respectively into mounting pockets formed in the chip-discharging pallet;
aligning the sensor chips by displacing the positions of the sensor chips mounted in the mounting pockets by use of a displacement mechanism provided to the measuring device;
fixing, by use of a fixing jig, the sensor chips aligned in the mounting pockets at the positions of the respective sensor chips; and
measuring the samples in the fixed sensor chips.

7. A chip-fixing device comprising:
a chip-fixing block having guides for mounting a plurality of sensor chips;
a displacement mechanism for displacing the positions of the respective sensor chips by moving the chip-fixing block, the displacement mechanism comprising
a cam mechanism including a first cam and a second cam, the first cam moving a first follower in one direction, the second cam being connected to the first follower and moving a second follower in a direction orthogonal to the direction in which the first follower moves, the second follower being connected to the chip-fixing block, and
a first linear guide bearing being connected to the second follower, and moving the chip-fixing block in a direction not orthogonal to the direction in which the second follower moves; and
a fixing jig for fixing the positions of the sensor chips displaced on the chip-Sing block, the fig jig comprising
a third follower moved by the first cam in the same direction as that in which the first follower moves,
a third cam connected to the third follower,
a fourth follower moved by the third cam in the same direction as that in which the third follower,
a second linear guide bearing allowing the fourth follower to move in a direction orthogonal to the sensor chips mounted on the chip-fixing block, and
chip-fixing pins connected to the fourth follower in an orthogonal direction, and are brought respectively into contact with the sensor chips so as to fix the sensor chips.

8. A chip-fig device according to claim 7, wherein
each of the guides is formed to hold three sides, including a shorter side, of the corresponding one of the sensor chips, and
a pair of the guides are formed respectively at such positions that parts, each holding one shorter side of the corresponding sensor chip, of the respective guides face each other.
